(19) United States
(12) Patent Application Publication
(10) Pub. No.: US 2014/0051658 A1
(43) Pub. Date: Feb. 20, 2014

(54) Chia Seed Mucilage As soothing Agent in Personal Care Products

(71) Applicant: Kemin Industries, Inc., Des Moines, IA (US)

(72) Inventor: Vandana Srivastava, Johnston, IA (US)

(73) Assignee: Kemin Industries, Inc., Des Moines, IA (US)

(21) Appl. No.: 13/966,756

(22) Filed: Aug. 14, 2013

Related U.S. Application Data
(60) Provisional application No. 61/682,821, filed on Aug. 14, 2012.

(51) Int. Cl. A61K 31/715

(52) U.S. Cl. CPC A61K 31/715
USPC 514/54; 536/123.1

(57) ABSTRACT

A composition for soothing the skin comprising mucilage extracted from chia seed (Salvia Hispanica L.).

The present application claims priority to U.S. Patent Application Ser. No. 61/682,821, filed Aug. 14, 2012, which is incorporated herein in its entirety by this reference.

### BACKGROUND OF THE INVENTION

The present invention relates generally to personal care products and, more specifically, to the use of mucilage extracted from chia seed as a soothing agent in personal care products.

Soothing agents are widely used in personal care in products for acne, sun protection, and post-severe treatments of skin and hair to counter the harsh effects of ingredients in personal care products. Soothing agents may be used in shampoos, soaps, toothpastes, laundry detergents and other personal and household care products that cause irritation. Upon exposure to water, chia seeds produce mucilage comprising a polysaccharide that may be the active agent in countering the effects of skin irritants. Extracts of chia seeds may also have immunomodulatory effects. There is a need for new soothing agents, particularly those obtained from natural sources.

### SUMMARY OF THE INVENTION

The invention consists of the use of mucilage extracted from chia seed as a soothing agent in personal care products. Chia seed are soaked in water until a mucilaginous exudate forms. When applied to irritated skin, a soothing effect similar to that of colloidal oatmeal was observed.

Chia seeds are obtained from the plant Salvia Hispanica L. The exudate from these seeds forms mucilage, polysaccharide in nature, when soaked in water. Extracted mucilage in the form of dried fluffy cotton was obtained. A controlled and blind Lactic Acid Sting Test (LAST) was conducted on a small group of people. The protocol was modified to exclude hill-top chambers considering the preliminary nature of the test material. This test was based on a sensory irritation initiated on the naso-labial fold of human skin by lactic acid. The most common reactions expected included stinging, itching and/or burning sensation. The test samples were applied on the irritated skin and a visual observation recorded. Hydrated dispersion of chia seed mucilage was tested with colloidal oatmeal as positive and deionized water as negative control.

A primary object of the present invention is to provide a botanical composition for soothing skin that will overcome the shortcomings of prior art compositions.

A further object of the present invention is to provide a botanical composition from chia seed for soothing skin which is able to soothe the pain and discomfort associated with skin reddening or erythema.

Another object of the present invention is to provide a botanical composition from chia seed for soothing skin which is able to be easily applied to the affected area.

A still further object of the present invention is to provide a botanical composition from chia seed for soothing skin which is in liquid form and able to be sprayed on the affected area.

A further object of the present invention is to provide a botanical composition from chia seed for soothing skin including all natural elements combined in a mixture.

Another object of the present invention is to provide a botanical composition from chia seed for soothing skin that is simple and easy to use.

A still further object of the present invention is to provide a botanical composition from chia seed for soothing skin that is economical in cost to manufacture.

### BRIEF DESCRIPTION OF THE FIGURES

FIG. 1 is a chart of the reaction of 12 subjects to the application of lactic acid to the skin of the naso-labial fold.

FIG. 2 is a chart of the reduction in redness after treatment application.

FIG. 3 is a chart of the response to burning sensation after application of treatments.

### DESCRIPTION OF THE INVENTION

### Materials and Methods

Chemicals and Equipment. Table 1 lists the chemicals and supplies used and Table 2 lists the equipment used in the preparation of chia seed mucilage.

TABLE 1
Chemicals and supplies used for preparation of Chia seed mucilage.

| Ingredients/ | Brand/Common | Grade/Source/ | Supplier/ |
|---|---|---|---|
| Supplies | Name | Certification | Manufacturer |
| Chia Seed | Alimentos | NA | The omega Tree, |
| | Ancestrales/Semillas | | Gilbert, AZ |
| | de Chia Seed | | |

TABLE 2
Equipment used for preparation of Chia seed mucilage

| Item | Manufacturer/Model |
|---|---|
| Stirring Plate | IKA RCT |
| Sonicator | Fisher Scientific 60 Sonic |
| | Dismembrator |
| Centrifuge | Sorvall Evolution RC |
| Vacuum Oven | Napco Model 5851 (E Series) |
| Freeze Drier | Virtis Sentry Freezemobile 12 SL |
| Weighing Scale | Mettler Toledo/New Classic SG |
| | 1502E/03 |

Preparation of Chia Seed Mucilage. Two samples of 25 g of whole chia seeds were placed in two glass beakers (2 L). One liter of 80° C. deionized water (pH˜4.74) was added (1:40 proportion) to each beaker. The mixtures were stirred with a magnetic stirrer at room temperature for three hours. The solutions were sonicated for five minutes at 10 Watts. The aqueous suspensions were centrifuged at 10,000 RPM for five minutes and the supernatants of both samples were combined (volume ˜1.4 L). The obtained seed cake was then spread on an aluminum foil tray and dried in an oven at 50° C. overnight. Once dry, the seed residue was put in plastic tubes (Corning 50 mL) and vortexed for several minutes in order to facilitate the dissociation of the left-over mucilage from the seeds. The solid residue was rehydrated with 80° C. deionized water (40 mL in five tubes), vortexed, allowed to swell for ten minutes and centrifuged at 10,000 RPM for five minutes. All the supernatants were collected and added to the first solution. The hydration process was repeated one more time. The homogenized solution (1.8 L) was aliquoted and freeze-dried. The fluffy solid residue was then collected (mass ˜6.55 g).

Chemicals and Equipment. Table 3 lists the chemicals and supplies used and Table 4 lists the equipment used in the preparation of samples and conducting the lactic acid sting test.

TABLE 3
Chemicals and supplies used for preparation of test material and
conducting the LAST.

| | Brand/ | Grade/ | |
|---|---|---|---|
| Ingredients/ | Common | Source/ | Supplier/ |
| Supplies | Name | Certification | Manufacturer |
| Chia Seed Mucilage | In-house | Experimental | In-house |
| (INCI name not | | | |
| determined) | | | |
| Avena Sativa (Oat) | Tech-O # 11- | USP/NF | Weeks & Leo Co. |
| Kernel Flour | 070/Colloidal | Category I | Inc./Beacon CMP |
| | Oatmeal | Skin | Corporation |
| | | Protectant | |
| L(+) Lactic Acid | Purac ® | FCC | Weeks & Leo Co. |
| | FCC 88 | | Inc., Des Moines, |
| | | | IA/Purac |
| | | | America, Inc., |
| | | | Blair, NE |
| Deionized Water | NA | NA | In-house |
| Cotton Swabs | Equate | NA | Walgreens |
| | | | Stores, Inc., |
| | | | Urbandale, IA |
| Facial Cleansing Pads | NA | NA | Walgreens |
| | | | Stores, Inc., |
| | | | Urbandale, IA |
| 1% Hydrocortisone | Cortizone.10 | NA | Weeks & Leo |
| Anti-Itch Ointment | | | Outlet, |
| | | | Urbandale, IA |

TABLE 4
Equipment used to prepare test sample.

| Item | Manufacturer/Model |
|---|---|
| Multi-speed stirrer | IKA/RW20 Digital |
| Weighing scale | Adventurer ™ Pro/OHaus |
| Digital Camera | Canon Powershot SD 780 IS Digital |
| | Elph |

Preparation of samples. To prepare the test sample, 0.5 g of chia seed mucilage was weighed in a disposable weighing boat. In a glass beaker (250 mL), 50 g water was weighed. This beaker was placed under the multi-speed stirrer and the impeller was positioned in the center. At 800 RPM, a vortex was created in the liquid and the chia seed mucilage was slowly dispersed. This dispersion was mixed for 30 minutes. This was labeled as Treatment A. A similar dispersion was made with colloidal oatmeal powder. This was labeled as Treatment B. Deionized water was obtained from the lab. This was labeled as Treatment C.

Lactic Acid Sting Test. The following protocol was used to conduct the LAST. The volunteer was seated and was informed about the procedure. A facial cleansing pad soaked in water was used to cleanse the nostril, and the naso-labial fold. A clean cotton swab was soaked in lactic acid kept in a glass beaker (150 mL). This was gently applied on the both sides of the nostril. Time was noted for the onset of signs of sensory irritation. The volunteers were requested to rate their reaction on a numerical scale listed in Table 5. Then the treatment was applied with a soaked cotton swab on the left nostril and naso-labial fold of the volunteer. The type of treatment was not disclosed to the volunteer. The change in the sensory irritation was observed and recorded using a digital camera and a scale filled by the observer on a scale listed in Table 6. The volunteer was requested again to grade the change on a numerical scale listed in Table 7. The test area was cleansed again with soaked facial cleansing pad and a hydrocortisone cream applied if the volunteer needed it. Five people with A, four with B and three people were treated with treatment C.

TABLE 5
Numerical scale for sensory irritation after lactic acid application

| Attribute | Scale |
|---|---|
| Very Itchy | 5 |
| Tolerable itchy | 4 |
| Itchy | 3 |
| Minor tingling/scratchy | 2 |
| No reaction | 1 |

TABLE 6
Numerical scale for change in redness (erythema) after treatment
application

| | |
|---|---|
| | Number of |
| Attribute | volunteers |

Did not develop redness
No difference at all
Very little difference
Most of it is gone
Completely gone

TABLE 7
Numerical scale for sensory irritation after treatment application

| Attribute | Scale |
|---|---|
| Completely soothed | 5 |
| Most of it is soothed | 4 |
| Some of it is soothed | 3 |
| Feels better but still itchy | 2 |
| Still as itchy as before | 1 |

### Results

Test samples. The dried extract of chia seed mucilage was obtained as fluffy cotton like powder. The hydrated dispersion of chia seed mucilage was translucent and light brown in color. It was thicker than water with a non-distinctive odor. The hydrated dispersion of colloidal oatmeal was white in color and the particles settled to the bottom. The particles had to be re-suspended before application.

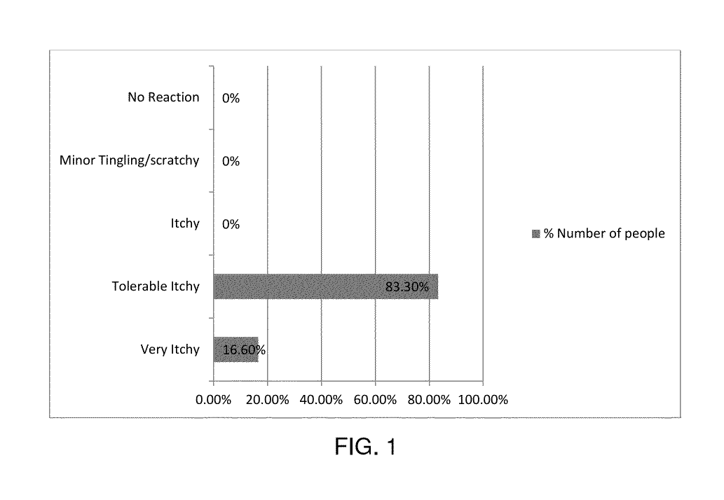

Reaction to Lactic Acid application. Ten out of twelve subjects reported the onset of tolerable itchiness and burning sensation within the first five minutes of application. Two subjects reported very itchy skin immediately after application (FIG. 1). Redness or erythema did not develop in half of the number of subjects.

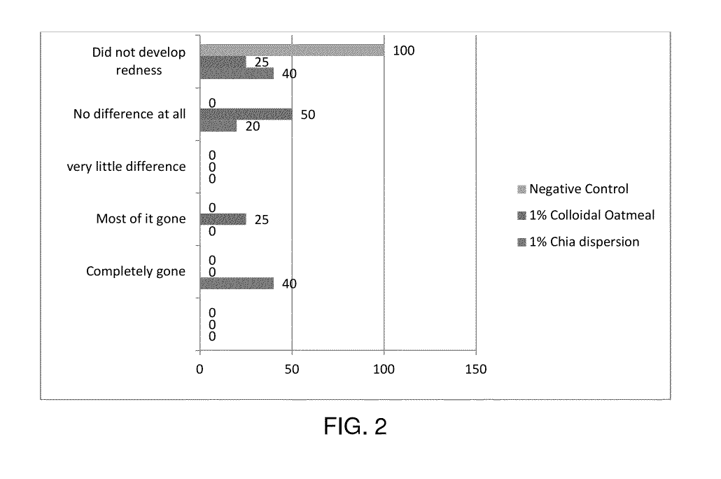

Reaction to Treatments. Redness did not appear in half of the subjects (FIG. 2). The redness was almost completely gone in two subjects applied with Treatment A and not much change was observed in the rest of them (FIG. 2).

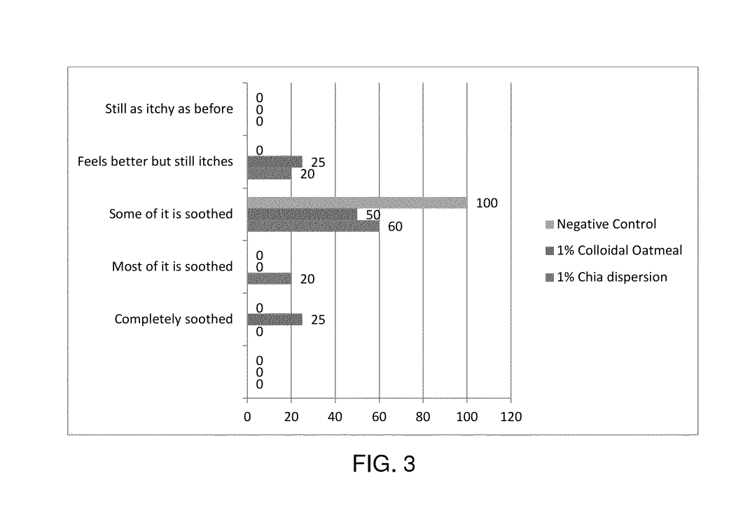

All the subjects treated with negative control felt that some of the burning sensation was soothed (FIG. 3). One subject felt complete alleviation of burning sensation after application of positive control, whereas 50% felt that it was somewhat soothed. 50% of subjects treated with chia seed mucilage responded that some of the burning sensation was soothed.

### Discussion

An experiment with chia seed mucilage was conducted to determine probable use as a soothing agent by virtue of its polysaccharide content. Chia seed mucilage was seen to reduce the appearance of redness as a response to sensory irritation induced by lactic acid. The response to burning sensation was comparable to water and colloidal oatmeal. These results indicate that chia seed mucilage shows potential benefits in applications in personal care products as soothing agents that aid in the reduction of redness.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A composition for soothing the skin, comprising mucilage extracted from chia seed (Salvia Hispanica L.).

2. A method of preparing a composition for soothing the skin, comprising the steps of:
(a) soaking chia seeds in water resulting in exudation of mucilage; and
(b) separating the exudated mucilage from the solid remnants of the chia seeds.

3. A method as defined in claim 2, wherein the separating step comprises drying the exudated mucilage and the solid remnants of the chia seeds and disassociating the dried mucilage from the solid remnants of the chia seeds, and further comprising rehydration of the dried mucilage.

4. A method of soothing skin, comprising
(a) soaking chia seeds in water resulting in exudation of mucilage;
(b) separating the exudated mucilage from the solid remnants of the chia seeds; and
(c) applying the separated mucilage to the skin.

5. A method as defined in claim 4, further comprising the step of adding additional skin-safe compounds to the mucilage prior to application to the skin.
